(19) United States
(12) Patent Application Publication
(10) Pub. No.: US 2024/0382067 A1
(43) Pub. Date: Nov. 21, 2024

(54) MEDICAL ASSISTANCE SYSTEM AND MEDICAL ASSISTANCE METHOD

(71) Applicant: OLYMPUS MEDICAL SYSTEMS CORP., Tokyo (JP)

(72) Inventors: Megumi NAGASAWA, Tokyo (JP); Haruhiko SAKAYORI, Tokyo (JP); Emi MASUDA, Tokyo (JP)

(73) Assignee: OLYMPUS MEDICAL SYSTEMS CORP., Tokyo (JP)

(21) Appl. No.: 18/786,983

(22) Filed: Jul. 29, 2024

Related U.S. Application Data
(63) Continuation of application No. PCT/JP2022/003615, filed on Jan. 31, 2022.

(51) Int. Cl. A61B 1/00; G06T 7/00; G06T 7/73; G06T 11/60; G06V 10/774

(52) U.S. Cl. CPC A61B 1/000094; A61B 1/000096; A61B 1/0005; G06T 7/0012; G06T 7/73; G06T 11/60; G06V 10/774; G06T 2200/24; G06T 2207/10068; G06T 2207/20081; G06T 2210/41; G06V 2201/031

(57) ABSTRACT

A first mark-imparting unit acquires presence information indicating that there is a biopsy spot in an endoscopic image and site information indicating a site included in the endoscopic image, and provides a first mark indicating that a biopsy has been performed or a biopsy should be performed to a position corresponding to the biopsy spot in an organ model image based on the presence information and the site information. A display image generation unit displays the organ model image provided with the first mark on a display device.

### CROSS-REFERENCE TO RELATED APPLICATIONS

This application is based upon and claims the benefit of priority from the International Application No. PCT/JP2022/003615, filed on Jan. 31, 2022, the entire contents of which are incorporated.

### BACKGROUND

### 1. Technical Field

The present disclosure relates to a medical assistance system and a medical assistance method for marking organ model images such as schema diagrams.

### 2. Description of the Related Art

In endoscopic examination, a doctor observes endoscopic images displayed on a display device and, upon finding a lesion, operates an endoscope release switch to capture (save) endoscopic images of the lesion. At this time, the doctor may use a treatment tool called forceps so as to remove a portion of the diseased tissue. Removing a portion of diseased tissue during an endoscopic examination is called “biopsy,” and the diseased tissue collected through biopsy is sent for pathological testing to serve as material for a definitive diagnosis.

JP 2010-282355A discloses a report system that enables a doctor to mark a sample removal position on an endoscopic image captured during an upper endoscopic examination, and to mark the sample removal position on a schema diagram that shows the shape of the observed organ in a lower endoscopic examination.

In the report creation work for an examination in which a biopsy has been performed, a doctor provides an endoscopic image of the biopsy spot with a biopsy mark indicating that a biopsy has been performed, and attaches the endoscopic image to the report. At this time, the doctor also provides a biopsy mark indicating that the biopsy has been performed to a position corresponding to the biopsy spot on a schema diagram included in the report. By providing a biopsy mark on the schema diagram, another doctor can immediately know the location of the biopsy spot in the examination when he or she looks at the schema diagram.

In the current report creation work, doctors manually provide biopsy marks to both endoscopic images and schema diagrams, which is time-consuming. Therefore, the development of a technology is desired that can efficiently assist report creation work.

### SUMMARY

In this background, a general purpose of the present disclosure is to provide a technology for automatically marking organ model images such as schema diagrams.

A medical assistance system according to one embodiment of the present disclosure includes: one or more processors comprising hardware, wherein the one or more processors are configured to: acquire presence information indicating that there is a biopsy spot in an endoscopic image and site information indicating a site included in the endoscopic image; provide a first mark indicating that a biopsy has been performed or a biopsy should be performed to a position corresponding to a biopsy spot in an organ model image based on the presence information and the site information; and display the organ model image provided with the first mark.

Another embodiment of the present disclosure relates to a medical assistance method including: acquiring presence information indicating that there is a biopsy spot in an endoscopic image; acquiring site information indicating a site included in the endoscopic image; providing a first mark indicating that a biopsy has been performed or a biopsy should be performed to a position corresponding to the biopsy spot in an organ model image based on the presence information and the site information; and displaying the organ model image provided with the first mark.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing the configuration of a medical assistance system according to an embodiment;

FIG. 2 is a diagram showing functional blocks of a server device;

FIG. 3 is a diagram showing functional blocks of an information processor;

FIG. 4 is a diagram showing an example of an organ model image;

FIG. 5 is a diagram showing a plurality of subregions constituting an organ model image;

FIG. 6 is a diagram showing an example of a report creation screen for inputting examination results;

FIG. 7 is a diagram showing an example of a selection screen for selecting endoscopic images;

FIG. 8 is a diagram showing an example of an editing screen for an endoscopic image;

FIG. 9 is a diagram showing a state in which a second biopsy mark is arranged on an endoscopic image;

FIG. 10 is a diagram showing an example of an editing screen for a schema diagram;

FIG. 11 is a diagram showing an example of an editing screen for another endoscopic image;

FIG. 12 is a diagram showing an example of an editing screen for another endoscopic image;

FIG. 13 is a diagram showing an example of a schema diagram editing screen;

FIG. 14 is a diagram showing an estimated direction of travel of an endoscope; and

FIG. 15 is a diagram showing an example of an editing screen for an endoscopic image including an organ model image.

### DETAILED DESCRIPTION

The disclosure will now be described by reference to the preferred embodiments. This does not intend to limit the scope of the present disclosure, but to exemplify the disclosure.

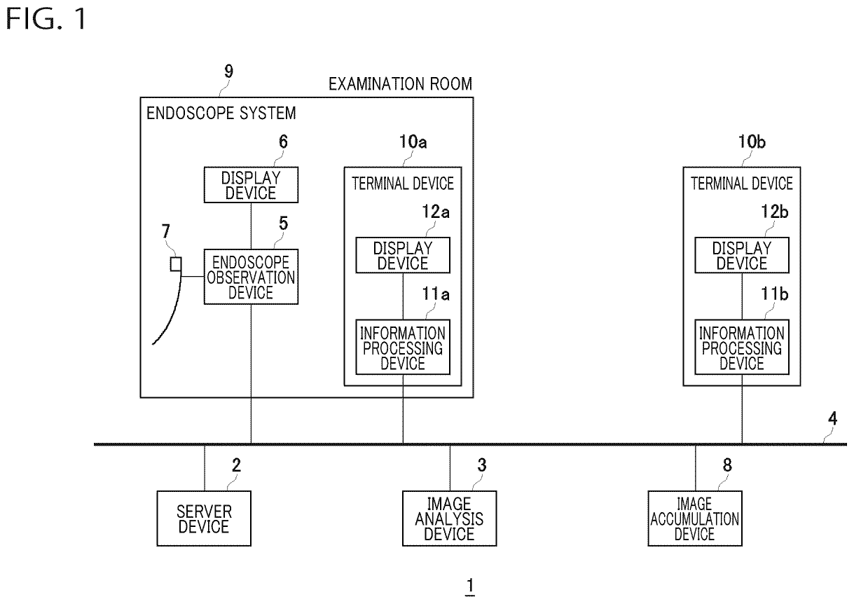

FIG. 1 shows the configuration of a medical assistance system 1 according to an embodiment. The medical assistance system 1 is provided in a medical facility such as a hospital where endoscopic examinations are performed. In the medical assistance system 1, a server device 2, an image analysis device 3, an image accumulation device 8, an endoscope system 9, and a terminal device 10b are communicably connected via a network 4 such as a local area network (LAN). The endoscope system 9 is installed in an examination room and has an endoscope observation device 5 and a terminal device 10a. In the medical assistance system 1, the server device 2, the image analysis device 3, and the image accumulation device 8 may be provided outside the medical facility, for example, in a cloud server.

The endoscope observation device 5 is connected to an endoscope 7 to be inserted into the digestive tract of a patient. The endoscope 7 has a light guide for illuminating the inside of the digestive tract by transmitting illumination light supplied from the endoscope observation device 5, and the distal end of the endoscope 7 is provided with an illumination window for emitting the illumination light transmitted by the light guide to living tissue and an imaging unit for imaging the living tissue at a predetermined cycle and outputting an imaging signal to the endoscope observation device 5. The imaging unit includes a solid-state imaging device, e.g., a CCD image sensor or a CMOS image sensor, that converts incident light into an electric signal.

The endoscope observation device 5 performs image processing on the imaging signal photoelectrically converted by a solid-state imaging device of the endoscope 7 so as to generate an endoscopic image and displays the endoscopic image on the display device 6 in real time. In addition to normal image processing such as A/D conversion and noise removal, the endoscope observation device 5 may include a function of performing special image processing for the purpose of highlighting, etc. The endoscope observation device 5 generates endoscopic images at a predetermined cycle, e.g., 1/60 seconds. The endoscope observation device 5 may be formed by one or more processors with dedicated hardware or may be formed by one or more processors with general-purpose hardware. The endoscope 7 according to the embodiment is a flexible endoscope and has a forceps channel for inserting an endoscopic treatment tool. By inserting biopsy forceps into the forceps channel and operating the inserted biopsy forceps, the doctor can perform a biopsy during an endoscopic examination and remove a portion of the diseased tissue.

According to the examination procedure, the doctor observes an endoscopic image displayed on the display device 6. The doctor observes the endoscopic image while moving the endoscope 7, and operates the release switch of the endoscope 7 when a biological tissue to be captured appears on the display device 6. The endoscope observation device 5 captures (saves) an endoscopic image at the time when the release switch is operated and transmits the captured endoscopic image to the image accumulation device 8 along with information identifying the endoscopic image (image ID). The endoscope observation device 5 may assign an image ID including a serial number to an endoscopic image in the order of capturing. The endoscope observation device 5 may transmit a plurality of captured endoscopic images all at once to the image accumulation device 8 after the examination is completed. The image accumulation device 8 records the endoscopic images transmitted from the endoscope observation device 5 in association with an examination ID for identifying the endoscopic examination.

In the embodiment, “imaging” refers to operation of converting incident light into an electrical signal performed by a solid-state image sensor of an endoscope 7, and “capturing” refers to operation of saving (recording) an endoscopic image generated by the endoscope observation device 5. The “imaging” may include an operation of generating an endoscopic image from the converted electrical signal performed by the endoscope observation device 5.

The terminal device 10a is installed in the examination room with an information processing device 11a and a display device 12a. The terminal device 10a may be used by doctors, nurses, and others in order to check information on biological tissue being imaged in real time during endoscopic examinations.

The terminal device 10b is provided with an information processing device 11b and a display device 12b and installed in a room other than the examination room. The terminal device 10b is used when a doctor creates a report of an endoscopic examination. The terminal devices 10a and 10b may be formed by one or more processors having general-purpose hardware in the medical facility.

In the medical assistance system 1 according to the embodiment, the endoscope observation device 5 displays endoscopic images in real time through the display device 6, and provides the endoscopic images along with meta information of the images to the image analysis device 3 in real time. The meta information may be information that includes at least the frame number and imaging time information of each image, where the frame number indicates the number of the frame after the endoscope 7 starts imaging.

The image analysis device 3 is an electronic calculator (computer) that analyzes endoscopic images to detect lesions in the endoscopic images and performs qualitative diagnosis of the detected lesions. The image analysis device 3 may be a computer-aided diagnosis (CAD) system with an artificial intelligence (AI) diagnostic function. The image analysis device 3 may be formed by one or more processors with dedicated hardware or may be formed by one or more processors with general-purpose hardware.

The image analysis device 3 may use a trained model that is generated by machine learning using endoscopic images for learning, information indicating organs and sites included in the endoscopic images, and information concerning lesion areas contained in the endoscopic images as training data. Annotation work on the endoscopic images is performed by annotators with expertise, such as doctors, and machine learning may use CNN, RNN, LSTM, etc., which are types of deep learning. Upon input of an endoscopic image, this trained model outputs information indicating an imaged organ, information indicating an imaged site, and information concerning an imaged lesion (lesion information). The lesion information output by the image analysis device 3 includes at least information on the presence or absence of a lesion indicating whether the endoscopic image contains a lesion or not. When the lesion is contained, the lesion information may include information indicating the size of the lesion, information indicating the location of the outline of the lesion, information indicating the shape of the lesion, information indicating the invasion depth of the lesion, and a qualitative diagnosis result of the lesion. The qualitative diagnostic result of the lesion includes the type of lesion. During an endoscopic examination, the image analysis device 3 is provided with endoscopic images from the endoscope observation device 5 in real time and outputs information indicating the organ, information indicating the site, and lesion information for each endoscopic image. Hereinafter, information indicating an organ, information indicating a site, and lesion information that are output for each endoscopic image are collectively referred to as “image analysis information.”

When the user operates the release switch (capture operation), the endoscope observation device 5 provides the frame number, imaging time, and image ID of the captured endoscopic image to the image analysis device 3, along with information indicating that the capture operation has been performed (capture operation information). Upon acquiring the capture operation information, the image analysis device 3 provides the image ID, the frame number, the imaging time information, and image analysis information for the provided frame number to the server device 2 along with the examination ID. The image ID, the frame number, the imaging time information, and the image analysis information constitute “additional information” that expresses the features and properties of the endoscopic image. Upon acquiring the capture operation information, the image analysis device 3 transmits the additional information to the server device 2 along with the examination ID, and the server device 2 records the additional information in association with the examination ID.

When the user finishes the endoscopic examination, the user operates an examination completion button on the endoscope observation device 5. The operation information of the examination completion button is provided to the server device 2 and the image analysis device 3, and the server device 2 and the image analysis device 3 recognize the completion of the endoscopic examination.

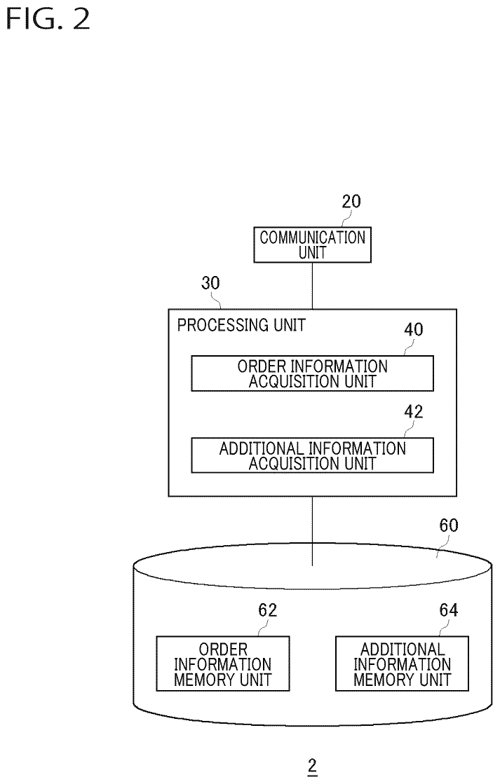

FIG. 2 shows functional blocks of the server device 2. The server device 2 includes a communication unit 20, a processing unit 30, and a memory device 60. The communication unit 20 transmits and receives information such as data and instructions between the image analysis device 3, the endoscope observation device 5, the image accumulation device 8, the terminal device 10a, and the terminal device 10b through the network 4. The processing unit 30 has an order information acquisition unit 40 and an additional information acquisition unit 42. The memory device 60 has an order information memory unit 62 and an additional information memory unit 64.

The server device 2 includes a computer. Various functions shown in FIG. 2 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 2 are realized by cooperation between hardware and software. Therefore, a person skilled in the art should appreciate that there are many ways of accomplishing these functional blocks in various forms in accordance with the components of hardware only, software only, or the combination of both.

The order information acquisition unit 40 acquires order information for an endoscopic examination from a hospital information system. For example, before the start of the examination work for one day at the medical facility, the order information acquisition unit 40 acquires the order information for the day from the hospital information system and stores the order information in the order information memory unit 62. Before the start of the examination, the endoscope observation device 5 or the information processing device 11a may read out order information for the examination to be performed from the order information memory unit 62 and display the order information on the display device.

The additional information acquisition unit 42 acquires the examination ID and additional information for the endoscopic image from the image analysis device 3, and stores the additional information in association with the examination ID in the additional information memory unit 64. The additional information for the endoscopic image includes an image ID, a frame number, imaging time information, and image analysis information.

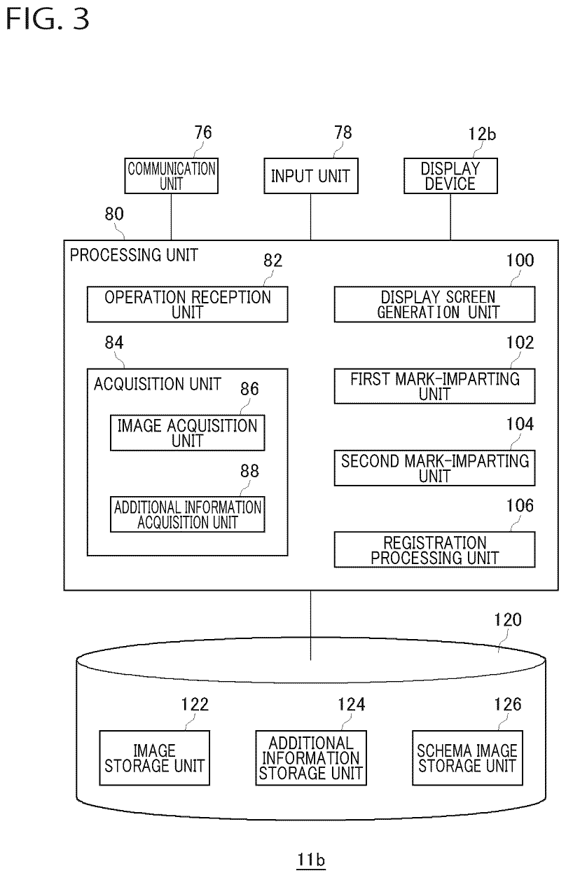

FIG. 3 shows the functional blocks of the information processing device 11b. The information processing device 11b has the function of assisting report creation work for an examination in which a biopsy has been performed and includes a communication unit 76, an input unit 78, a processing unit 80, and a memory device 120. The communication unit 76 transmits and receives information such as data and instructions between the server device 2, the image analysis device 3, the endoscope observation device 5, the image accumulation device 8, and the terminal device 10a via the network 4. The processing unit 80 includes an operation reception unit 82, an acquisition unit 84, a display screen generation unit 100, a first mark-imparting unit 102, a second mark-imparting unit 104, and a registration processing unit 106, and the acquisition unit 84 has an image acquisition unit 86 and an additional information acquisition unit 88. The memory device 120 has an image storage unit 122, an additional information storage unit 124, and a schema image storage unit 126.

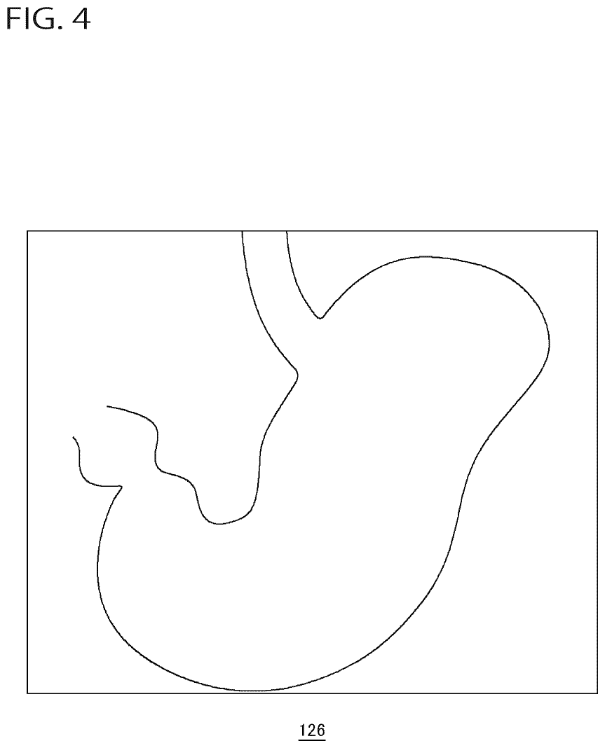

FIG. 4 shows an example of an organ model image stored in the schema image storage unit 126. The schema image storage unit 126 stores a schema diagram schematically representing the shape of an observed organ as an organ model image. The schema diagram is attached to an examination report to represent the location of a biopsy spot in the organ, and FIG. 4 shows a schema diagram of the stomach. The schema image storage unit 126 may store a schema diagram of the esophagus or duodenum, which are other observed organs in an upper endoscopic examination, and a schema diagram of the large intestine in a lower endoscopic examination.

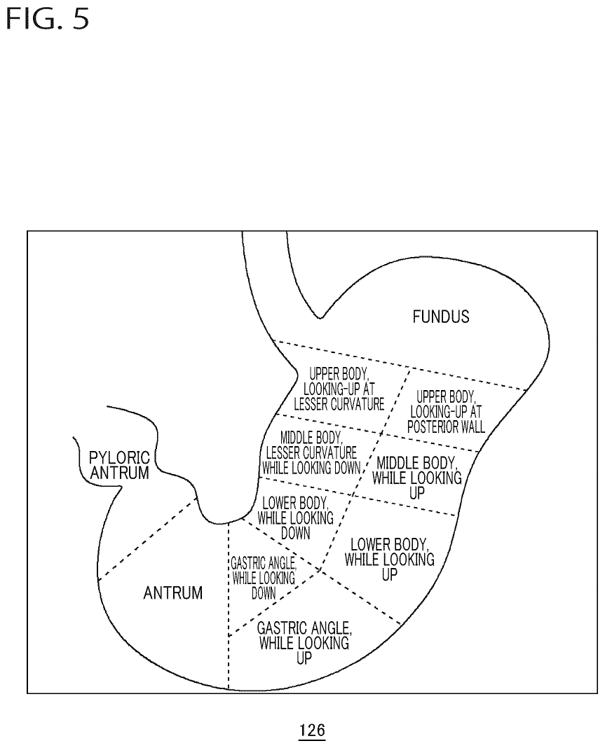

FIG. 5 shows a plurality of subregions constituting an organ model image. In an embodiment, a schema diagram of the stomach is divided into a plurality of subregions. As described above, machine learning is performed such that when an endoscopic image is input, a trained model in the image analysis device 3 outputs information indicating an imaged site (the site included in the endoscopic image). However, an organ model image is preferably divided into a plurality of subregions corresponding to a plurality of sites output by the image analysis device 3. That is, in the embodiment, one organ is divided into a plurality of sites, the schema diagram of the organ is divided into a plurality of subregions, and one site of the organ is associated with one subregion in the schema diagram. In the embodiment, the “stomach,” which is an organ, is divided into a plurality of sites: “fundus”; “upper body, looking-up at lesser curvature”; “upper body, looking-up at posterior wall”; “middle body, lesser curvature while looking down”; “middle body, while looking up”; “lower body, while looking down”; “lower body, while looking up”; “gastric angle, while looking down”; “gastric angle, while looking up”; “antrum”; and “pyloric antrum,” and the subregions shown in FIG. 5 are set according to this division.

The information processing device 11b includes a computer. Various functions shown in FIG. 3 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 3 are realized by cooperation between hardware and software. Therefore, a person skilled in the art should appreciate that there are many ways of accomplishing these functional blocks in various forms in accordance with the components of hardware only, software only, or the combination of both.

After the completion of an endoscopic examination, the user, a doctor, inputs a user ID and a password to the information processing device 11b so as to log in. An application for creating an examination report is activated when the user logs in, and a list of already performed examinations is displayed on the display device 12b. The list of already performed examinations displays examination information such as a patient name, a patient ID, examination date and time, an examination item, and the like in a list, and the user operates the input unit 78 such as a mouse or a keyboard so as to select an examination for which a report is to be created. When the operation reception unit 82 receives an examination selection operation, the image acquisition unit 86 acquires a plurality of endoscopic images linked to the examination ID of the examination selected by the user from the image accumulation device 8 and stores the endoscopic images in the image storage unit 122, and the additional information acquisition unit 88 acquires additional information linked to the examination ID of the examination selected by the user from the server device 2 and stores the additional information in the additional information storage unit 124. The display screen generation unit 100 generates a report creation screen and displays the report creation screen on the display device 12b.

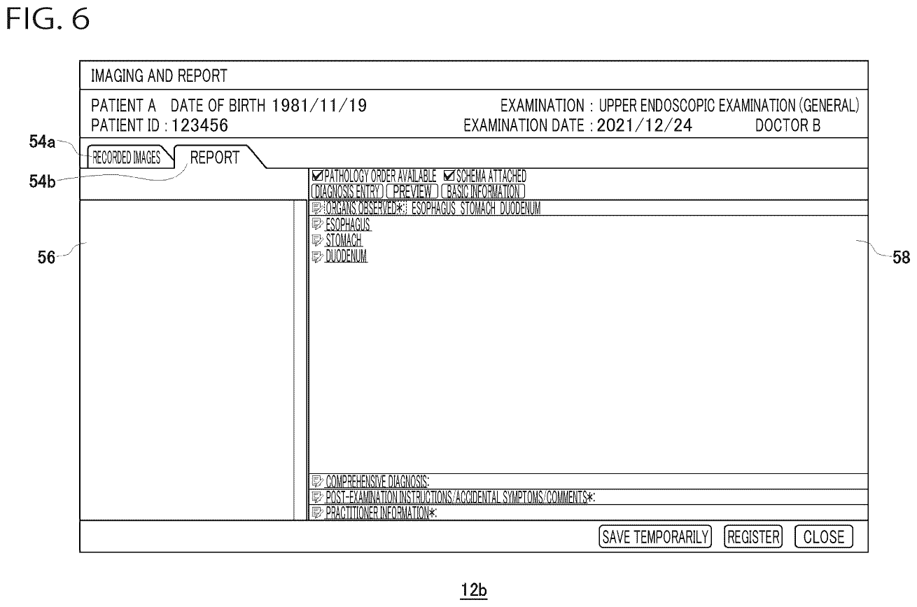

FIG. 6 shows an example of a report creation screen for inputting examination results. The report creation screen is displayed on the display device 12b while a report tab 54b is being selected. In the upper part of the screen, information such as a patient name, a patient ID, the date of birth, examination, an examination date, and a performing doctor is displayed. These pieces of information are included in the examination order information and may be acquired from the server device 2. In the embodiment, it is assumed that a biopsy has been performed in an examination for which a report is to be created. The user operates the input unit 78 and selects check boxes for both “pathology order available” and “schema attached,” and check marks indicating that the check boxes are being selected are displayed on both check boxes.

The report creation screen includes two regions: an attached image display region 56 for displaying endoscopic images to be attached in a region on the left side; and an input region 58 for the user to input the examination results in a region on the right side. In the input region 58, an area is provided for entering diagnosis details for “esophagus,” “stomach,” and “duodenum,” which are observation ranges in an upper endoscopic examination. The input region 58 may have a format where a plurality of selections are displayed for examination results such that the user enters a diagnosis detail by selecting a check box or may have a free format for free text entry.

The attached image display region 56 is a region for displaying endoscopic images to be attached to a report side by side. The user selects an endoscopic image to be attached to the report from an endoscopic image selection screen. The user selects a recorded image tab 54a to display endoscopic images captured in the examination on the display device 12b in a list.

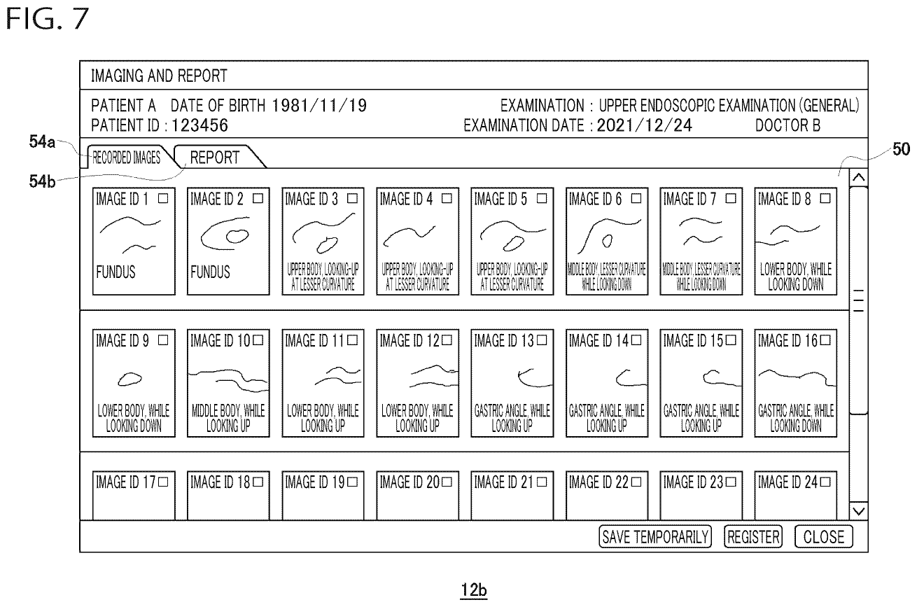

FIG. 7 shows an example of a selection screen for selecting endoscopic images to be attached to a report. The selection screen for endoscopic images is displayed on the display device 12b while the recorded image tab 54a is being selected. The display screen generation unit 100 generates a selection screen in which a plurality of endoscopic images captured by the user are arranged according to the imaging order, and displays the selection screen in a list display region 50. In the list display region 50, the endoscopic images may be displayed as reduced thumbnail images. The display screen generation unit 100 may refer to the additional information of each endoscopic image and display the image ID of the endoscopic image and a site name indicating the site included in the endoscopic image along with the endoscopic image.

The endoscopic images displayed in the list display region 50 are each provided with a check box. When the user operates a mouse to place a mouse pointer on a check box followed by left-clicking, the operation reception unit 82 receives the operation as an operation to select the corresponding endoscopic image as an attached image for a report, and the endoscopic image is selected as the attached image for the report. Endoscopic images selected as report attachment images are displayed side by side in the attached image display region 56 (see FIG. 6) when a report creation screen is displayed.

In the list display region 50, when the user places the mouse pointer on an endoscopic image and performs a predetermined operation, the operation reception unit 82 receives the operation as an operation to display the endoscopic image in an enlarged manner. The display screen generation unit 100 generates a display screen including the endoscopic image enlarged in an editable manner, and displays the display screen on the display device 12b. The user operation instructing the enlarged display may be a double-clicking operation for the endoscopic image. The user performs an operation of double-clicking an endoscopic image with an image ID 3 in which the biopsy spot is imaged.

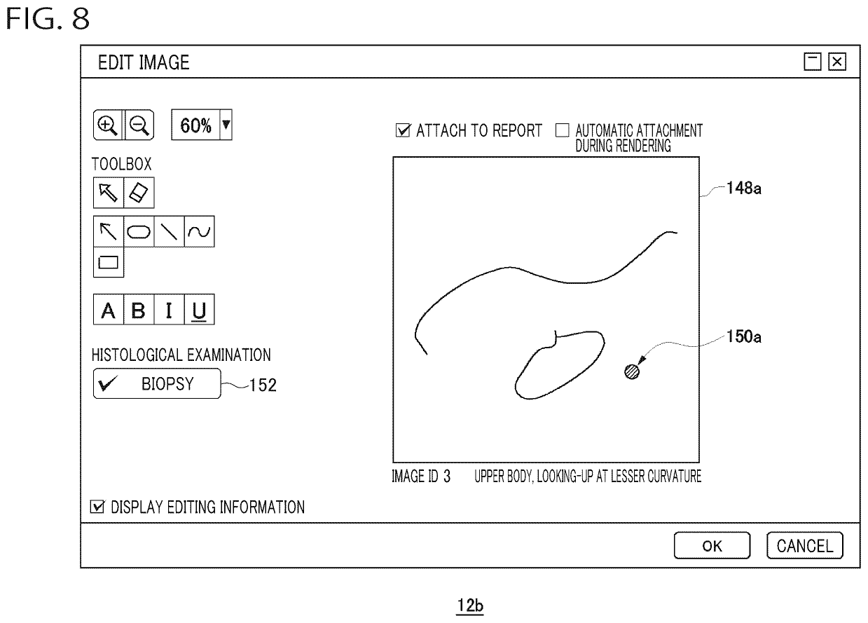

FIG. 8 shows an example of an editing screen for an endoscopic image. When the user performs an operation of double-clicking the endoscopic image with the image ID 3 in the list display region 50, the display screen generation unit 100 generates a display screen including an endoscopic image 148a enlarged in an editable manner and displays the display screen on the display device 12b. Displaying the endoscopic image in an enlarged manner allows the user to easily confirm a biopsy spot 150a included in the endoscopic image 148a. The biopsy spot 150a may be a diseased tissue imaged before performing a biopsy, or may be a diseased tissue imaged after performing a biopsy. For example, if the doctor captures the endoscopic image immediately before performing the biopsy, the biopsy spot 150a is the location of the diseased tissue immediately before being removed, and if the doctor captures the endoscopic image immediately after performing the biopsy, the biopsy spot 150a is the location of the diseased tissue that has already been collected. The biopsy spot 150a may be a location where the doctor determines a biopsy should be performed.

When the user presses a biopsy designation button 152, the second mark-imparting unit 104 enters a state of being able to provide the enlarged endoscopic image 148a with a second biopsy mark indicating that a biopsy has been performed or that a biopsy should be performed in accordance with the user operation. In the case of a mouse operation, when the user places the mouse pointer at a position near the biopsy spot 150a included in the endoscopic image 148a and performs a predetermined operation, the operation reception unit 82 receives the predetermined operation as an operation for placing a second biopsy mark at the position of the mouse pointer. Therefore, the second biopsy mark indicates the position where a biopsy has been performed or the position at which a biopsy should be performed in the endoscopic image 148a.

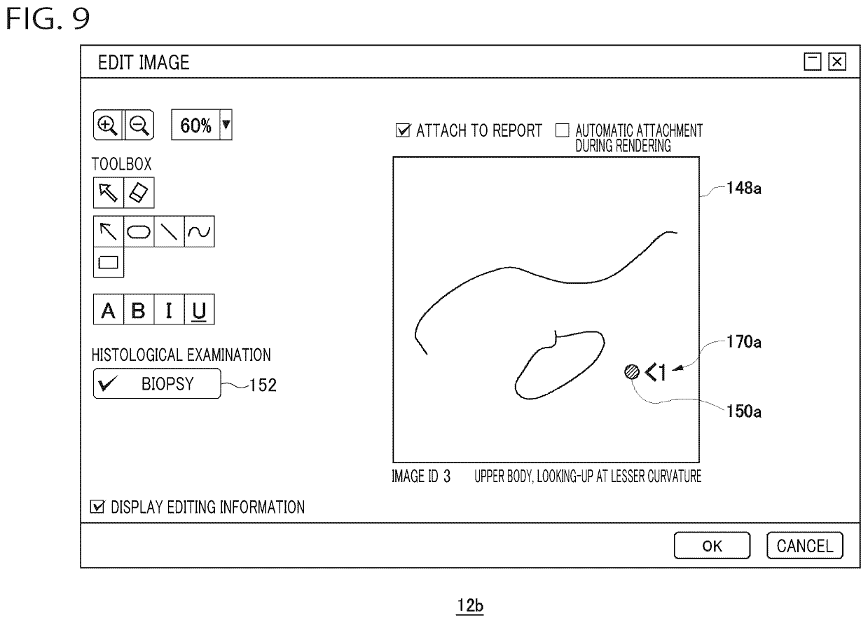

FIG. 9 shows a state in which a second biopsy mark 170a is arranged on the endoscopic image 148a. When the operation reception unit 82 receives a user operation of placing the second biopsy mark 170a on the endoscopic image 148a, the second mark-imparting unit 104 provides a second biopsy mark 170a at a position designated by the user operation.

In the example shown in FIG. 9, the second biopsy mark 170a “<1” is provided near the biopsy spot 150a. The number included in a second biopsy mark indicates the order in which the user marked a biopsy spot. Therefore, the first-provided second biopsy mark is “<1,” and the second-provided second biopsy mark is “<2.” The second mark-imparting unit 104 counts the number of second biopsy marks that have been provided, and sets the second biopsy marks to have consecutive numbers.

The information processing device 11b according to the embodiment has a function of automatically providing a first biopsy mark corresponding to a second biopsy mark 170a on a schema diagram when the second biopsy mark 170a is provided on the endoscopic image 148a. The first biopsy mark corresponding to the second biopsy mark 170a is a biopsy mark indicating that a biopsy has been performed or should be performed. Therefore, the first biopsy mark indicates the position where a biopsy has been performed or should be performed in the schema diagram. When the user provides a second biopsy mark 170a on the endoscopic image 148a, the information processing device 11b automatically provides a first biopsy mark on the schema diagram, thereby allowing the user to be assisted in efficient report creation work.

More specifically, when the second mark-imparting unit 104 provides a second biopsy mark 170a to the endoscopic image 148a, the first mark-imparting unit 102 acquires presence information indicating the presence of a biopsy spot in the endoscopic image 148a and site information indicating a site included in the endoscopic image 148a. The presence information may include information indicating that there is a spot to be biopsied in the endoscopic image 148a, or information indicating that there is already a biopsied spot in the endoscopic image 148a. The presence information may be information indicating that a second biopsy mark 170a has been provided to the endoscopic image 148a.

The first mark-imparting unit 102 acquires site information associated with the image ID of the endoscopic image 148a. The additional information of the endoscopic image with the image ID 3 includes the site information “upper body, looking-up at lesser curvature.” Therefore, the first mark-imparting unit 102 recognizes that a biopsy spot exists in the “upper body, looking-up at lesser curvature” based on the presence information and the site information, and provides a first biopsy mark indicating that a biopsy has been performed or that a biopsy should be performed on the schema diagram. When the second mark-imparting unit 104 provides a second biopsy mark 170a to the endoscopic image 148a according to the user operation, the first mark-imparting unit 102 may immediately provide a first biopsy mark to the schema diagram. When the operation reception unit 82 receives a user operation of switching a display image to a schema diagram while a screen shown in FIG. 9 is being displayed on the display device 12b, the display screen generation unit 100 displays the schema diagram provided with the first biopsy mark on the display device 12b in place of the endoscopic image 148a.

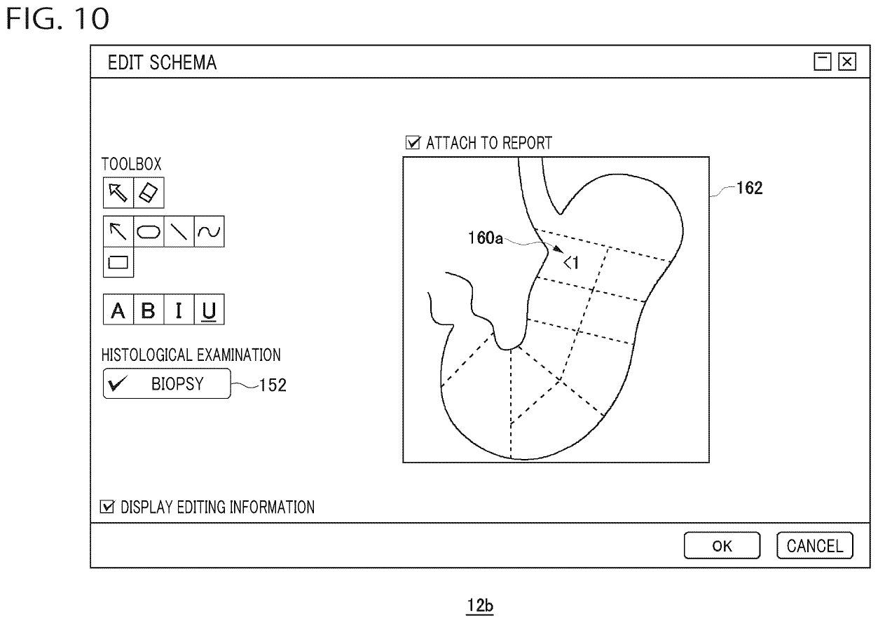

FIG. 10 shows an example of an editing screen for a schema diagram. Based on the provision of the second biopsy mark 170a to the endoscopic image 148a, the first mark-imparting unit 102 automatically provides a first biopsy mark 160a indicating that a biopsy has been performed or that a biopsy should be performed, at a position corresponding to the biopsy spot in the organ model image. The second biopsy mark 170a and the first biopsy mark 160a may be the same mark “<1.” The display screen generation unit 100 displays the organ model image provided with the first biopsy mark 160a on a schema editing screen. The first mark-imparting unit 102 automatically provides the first biopsy mark 160a to a subregion associated with “upper body, looking-up at lesser curvature” (see FIG. 5). The first mark-imparting unit 102 preferably arranges the first biopsy mark 160a in the substantial center of a subregion of the “upper body, looking-up at lesser curvature.” In the organ model image 162 shown in FIG. 10, site dividing lines are drawn. However, the dividing lines do not need to be drawn.

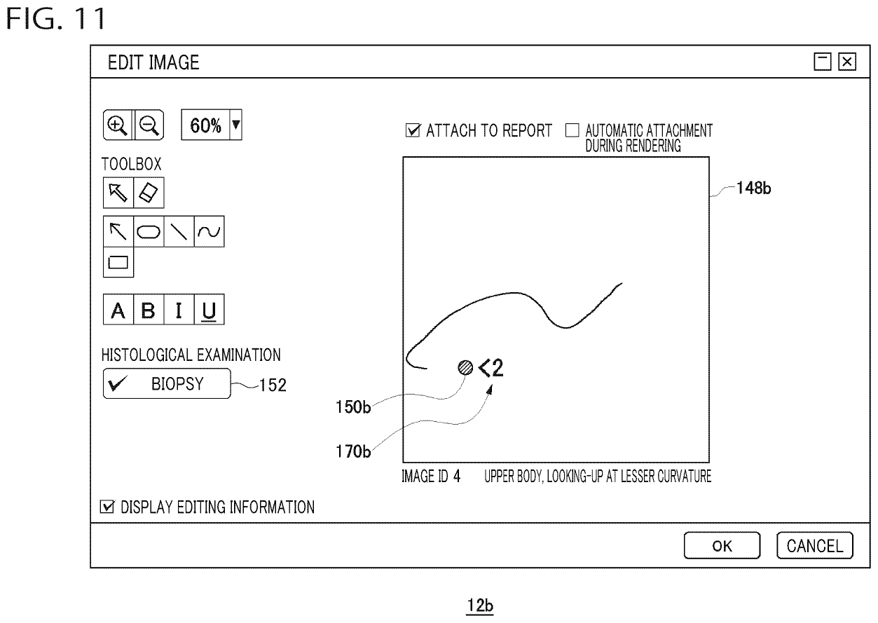

FIG. 11 shows an example of an editing screen for another endoscopic image. When the user performs an operation of double-clicking an endoscopic image with an image ID 4 in the selection screen (see FIG. 7), the display screen generation unit 100 generates a display screen including an endoscopic image 148b enlarged in an editable manner and displays the display screen on the display device 12b. Displaying the endoscopic image in an enlarged manner allows the user to easily confirm a biopsy spot 150b included in the endoscopic image 148b. The biopsy spot 150b may be a diseased tissue imaged before performing a biopsy, or may be a diseased tissue imaged after performing a biopsy.

When the user presses the biopsy designation button 152, the second mark-imparting unit 104 enters a state of being able to provide the enlarged endoscopic image 148b with a second biopsy mark indicating that a biopsy has been performed or that a biopsy should be performed in accordance with the user operation. When the operation reception unit 82 receives a user operation of placing a second biopsy mark on the endoscopic image 148b, the second mark-imparting unit 104 provides a second biopsy mark 170b at a position designated by the user operation. In the example shown in FIG. 11, the second biopsy mark 170b “<2” is provided near the biopsy spot 150b.

The first mark-imparting unit 102 acquires presence information indicating the presence of a biopsy spot in the endoscopic image 148b and site information indicating a site included in the endoscopic image 148b at this time. The first mark-imparting unit 102 acquires site information “upper body, looking-up at lesser curvature” associated with the image ID 4 of the endoscopic image 148b. Therefore, the first mark-imparting unit 102 recognizes that a biopsy spot exists in the “upper body, looking-up at lesser curvature” based on the presence information and the site information, and provides a first biopsy mark at a position corresponding to the biopsy spot in the schema diagram.

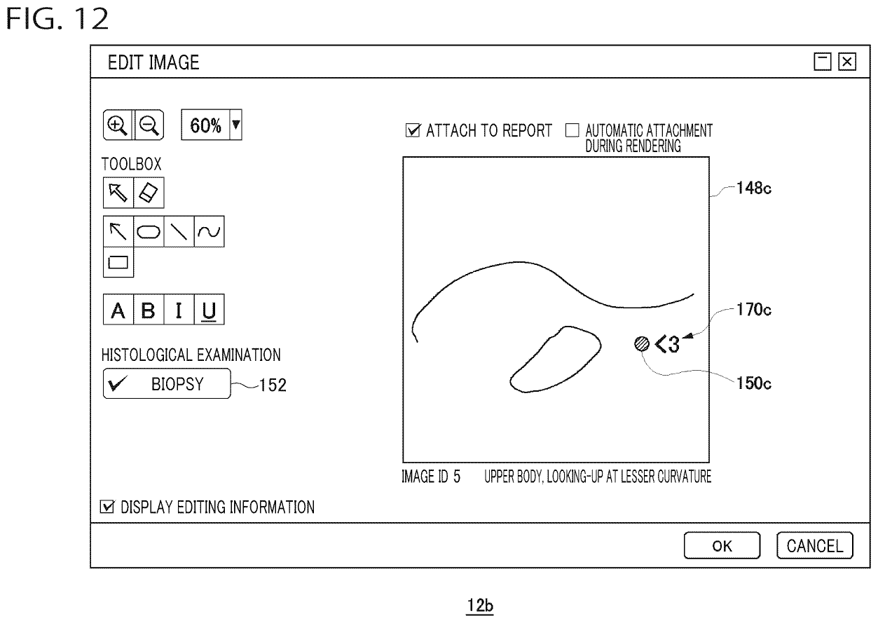

FIG. 12 shows an example of an editing screen for another endoscopic image. When the user performs an operation of double-clicking an endoscopic image with an image ID 5 in the selection screen (see FIG. 7), the display screen generation unit 100 generates a display screen including an endoscopic image 148c enlarged in an editable manner and displays the display screen on the display device 12b. When the operation reception unit 82 receives a user operation of placing a second biopsy mark on the endoscopic image 148c, the second mark-imparting unit 104 provides a second biopsy mark 170c at a position designated by the user operation. In the example shown in FIG. 12, the second biopsy mark 170c “<3” is provided near the biopsy spot 150c.

The first mark-imparting unit 102 acquires presence information indicating the presence of a biopsy spot in the endoscopic image 148c and site information indicating a site included in the endoscopic image 148c at this time. The first mark-imparting unit 102 acquires site information “upper body, looking-up at lesser curvature” associated with the image ID 5 of the endoscopic image 148c. Therefore, the first mark-imparting unit 102 recognizes that a biopsy spot exists in the “upper body, looking-up at lesser curvature” based on the presence information and the site information, and provides a first biopsy mark at a position corresponding to the biopsy spot in the schema diagram.

When the operation reception unit 82 receives a user operation of switching a display image to a schema diagram while a screen shown in FIG. 12 is being displayed on the display device 12b, the display screen generation unit 100 displays the schema diagram provided with a plurality of first biopsy marks in place of the endoscopic image 148c.

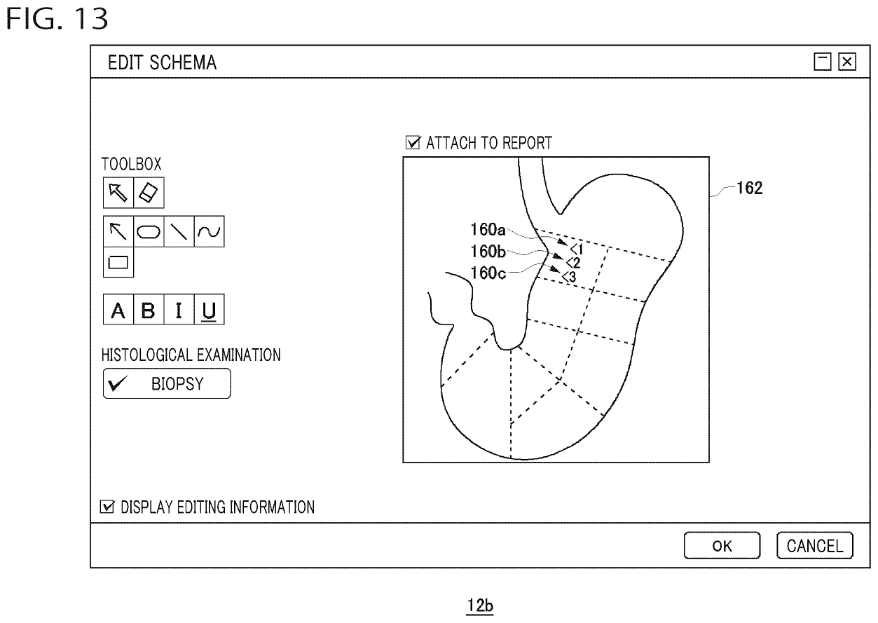

FIG. 13 shows an example of an editing screen for a schema diagram. The first mark-imparting unit 102 automatically provides first biopsy marks 160a, 160b, and 160c corresponding respectively to second biopsy marks 170a, 170b, and 170c provided to a plurality of endoscopic images at positions corresponding to biopsy spots in an organ model image. The first biopsy mark 160a may be the same “<1” as the second biopsy mark 170a, the first biopsy mark 160b may be the same “<2” as the second biopsy mark 170b, and the first biopsy mark 160c may be the same “<3” as the second biopsy mark 170c.

In an embodiment, the three endoscopic images with image IDs 3 to 5 are images of the same site (upper body, looking-up at lesser curvature), and each include a different biopsy spot. When there are a plurality of endoscopic images of different biopsy spots at the same site, the first mark-imparting unit 102 determines the position of the plurality of first biopsy marks 160a, 160b, and 160c in the subregion associated with the same site based on the imaging order.

More specifically, the first mark-imparting unit 102 acquires timing information indicating the imaging time of the three endoscopic images with the image IDs 3 to 5, and determines the imaging order of the three endoscopic images. The timing information may be the frame number or imaging time information of an image, or may simply be a serial number included in the image ID (assigned in the order of capturing). In this example, it is specified that the endoscopic image with the image ID 3, the endoscopic image with the image ID 4, and the endoscopic image with the image ID 5 have been captured in this order. The first mark-imparting unit 102 arranges the first biopsy marks 160a, 160b, and 160c side by side according to the imaging order such that the marks do not overlap in the subregion of the “upper body, looking-up at lesser curvature.”

Based on site information of each of a plurality of captured endoscopic images and timing information indicating the imaging time, the first mark-imparting unit 102 may estimate the travel direction of the endoscope in the endoscopic examination and determine the position of the first biopsy marks 160a, 160b, and 160c in the subregion of the “upper body, looking-up at lesser curvature” based on the estimated travel direction. The first mark-imparting unit 102 estimates the travel direction of the endoscope based on endoscope images including another site captured temporally before and after the endoscopic images with the image IDs 3 to 5 of the biopsy spot. In reference to FIG. 7, the endoscopic image with the image ID 2 captured temporally before is an image of “fundus,” and the endoscopic image with the image ID 6 captured temporally after is an image of “middle body, lesser curvature while looking down.” Therefore, the first mark-imparting unit 102 recognizes that the endoscope has moved in the order of “fundus” to “upper body, looking-up at lesser curvature” to “middle body, lesser curvature while looking down” so as to image each of the sites.

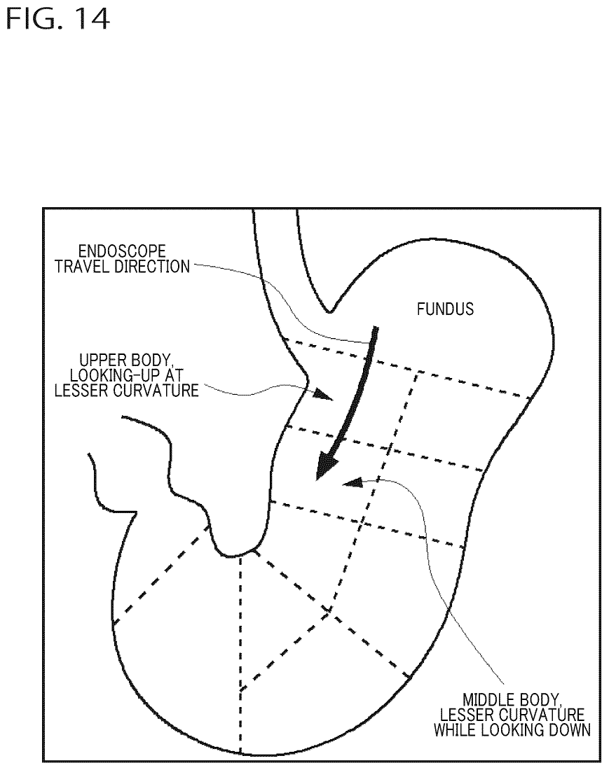

FIG. 14 shows an estimated direction of travel of the endoscope. The first mark-imparting unit 102 determines the position of the first biopsy marks 160a, 160b, and 160c in the subregion of the “upper body, looking-up at lesser curvature” according to the estimated travel direction of the endoscope and the imaging order of the three endoscopic images with the image IDs 3 to 5. More specifically, the first mark-imparting unit 102 places the first biopsy mark 160a near the entrance of the subregion of the “upper body, looking-up at lesser curvature” in the travel direction of the endoscope, places the first biopsy mark 160c near the exit of the subregion, and places the first biopsy mark 160b between the first biopsy marks 160a and 160c (see FIG. 13). By placing a plurality of first biopsy marks 160a, 160b, and 160c based on the travel direction of the endoscope and the imaging order, the first mark-imparting unit 102 allows the position of the first biopsy marks 160a, 160b, and 160c on the schema diagram to approximate the actual position of the biopsy spots 150a, 150b, and 150c.

In the report creation work, the user selects an image to be attached to a report, inputs the examination results in the input region 58 on the report creation screen, and creates the report. When the user operates a registration button (see FIG. 6), the registration processing unit 106 registers the details input on the report creation screen in the server device 2, and the report creation operation is completed.

Described above is an explanation based on the embodiment of the present disclosure. The embodiment is intended to be illustrative only, and it will be obvious to those skilled in the art that various modifications to constituting elements and processes could be developed and that such modifications are also within the scope of the present disclosure. In the embodiment, the endoscope observation device 5 transmits user-captured images to the image accumulation device 8. However, in an exemplary variation, the image analysis device 3 may transmit user-captured images to the image accumulation device 8. In the embodiment, the information processing device 11b has the processing unit 80. However, in the exemplary variation, the server device 2 may have the processing unit 80.

In the embodiment, the display screen generation unit 100 displays an organ model image provided with first biopsy marks and an endoscopic image provided with second biopsy marks on different screens. However, in an exemplary variation, an organ model image provided with first biopsy marks and an endoscopic image provided with second biopsy marks may be displayed on the same screen at the same time.

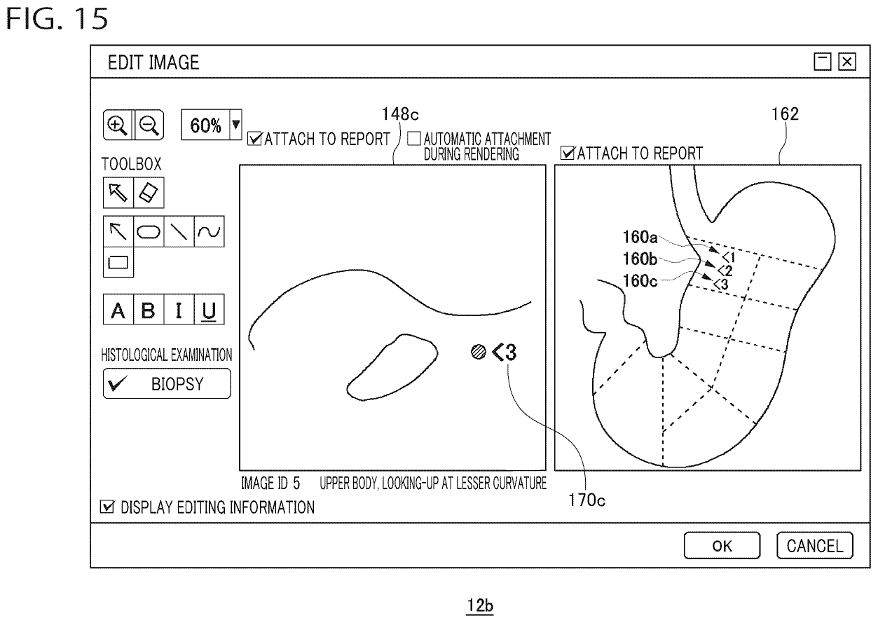

FIG. 15 shows an example of an editing screen for an endoscopic image including an organ model image 162. In this example, the display screen generation unit 100 displays the endoscopic image 148c with the image ID 5 and the organ model image 162 on the same screen. When the operation reception unit 82 receives a user operation of placing a second biopsy mark on the endoscopic image 148c and the second mark-imparting unit 104 provides a second biopsy mark 170c at a position designated by the user operation, the first mark-imparting unit 102 provides a first biopsy mark 160c corresponding to the second biopsy mark 170c to the organ model image 162. At this time, the first mark-imparting unit 102 may determine the placement positions of the plurality of first biopsy marks 160a, 160b, and 160c such that the first biopsy marks do not overlap based on the estimated travel direction of the endoscope and the imaging time of a plurality of biopsy spots at the same site. By displaying the endoscopic image 148c and the organ model image 162 on the same screen, the user can easily confirm that when the second biopsy mark 170c is provided to the endoscope image 148c, the first biopsy mark 160c corresponding to the second biopsy mark 170c is automatically provided on the organ model image 162.

In the embodiment, when the operation reception unit 82 receives a user operation of placing a second biopsy mark on the endoscope image 148c, the second mark-imparting unit 104 provides a second biopsy mark to the endoscopic image. In an exemplary variation, the second mark-imparting unit 104 may provide a second biopsy mark based on image analysis information output by the image analysis device 3.

In this exemplary variation, the image analysis device 3 has a function of detecting a biopsy spot included in an endoscopic image. The image analysis device 3 checks by image analysis whether or not a biopsy spot is included in the endoscopic image. For each endoscopic image, the image analysis device 3 outputs information indicating whether or not a biopsy spot is included, and, if a biopsy spot is included, outputs position information indicating the position of the biopsy spot. That is, when the image analysis device 3 recognizes that a biopsy spot is included in an endoscopic image, presence information indicating the existence of a biopsy spot in the endoscopic image and position information indicating the position of the biopsy spot (position coordinates in the endoscopic image). In this exemplary variation, this presence information and the position information are provided to the server device 2 as a part of image analysis information.

Thus, in the exemplary variation, the second mark-imparting unit 104 provides a second biopsy mark to the endoscopic image, and the first mark-imparting unit 102 provides a first biopsy mark to the organ model image at the same time based on the presence information and the position information output by the image analysis device 3. According to the exemplary variation, the provision of a second biopsy mark to an endoscopic image and the provision of a first biopsy mark to an organ model image may all be performed automatically, which can assist the user to further improve the efficiency of the report creation work.

What is claimed is:

1. A medical assistance system comprising:
one or more processors comprising hardware, wherein an organ model image includes a plurality of subregions associated with a plurality of sites of an organ, and wherein
the one or more processors are configured to:
acquire presence information indicating that there is a biopsy spot in an endoscopic image, site information indicating a site included in the endoscopic image, and timing information indicating an imaging time of the endoscopic image for each of a plurality of endoscopic images taken in one endoscopic examination;
provide a first mark indicating that a biopsy has been performed or a biopsy should be performed to the subregion in the organ model image associated with the site indicated by the site information based on the presence information and the site information;
when there are a plurality of the endoscopic images of a different biopsy spot in the same site, determine positions of a plurality of the first marks in the subregion associated with the same site based on an imaging order; and
display the organ model image provided with the plurality of the first marks.

2. The medical assistance system according to claim 1, wherein
the one or more processors are configured to:
provide a second mark indicating that a biopsy has been performed or a biopsy should be performed to the endoscopic image of the biopsy spot.

3. The medical assistance system according to claim 2, wherein
the one or more processors are configured to:
receive a user operation of placing the second mark on the endoscopic image; and
provide the second mark on the endoscopic image according to the user operation.

4. The medical assistance system according to claim 2, wherein
the one or more processors are configured to:
acquire the presence information and position information indicating the position of the biopsy spot based on the endoscopic image; and
provide the second mark to the endoscopic image based on the presence information and the position information.

5. The medical assistance system according to claim 1, wherein
the one or more processors are configured to:
acquire the site information based on the endoscopic image.

6. The medical assistance system according to claim 5, wherein
the one or more processors are configured to:
input the endoscopic image into a trained model and acquire the site information output from the trained model, wherein
the trained model is generated by machine learning using endoscopic images for learning and information indicating a site included in the endoscopic images as training data.

7. The medical assistance system according to claim 1, wherein
the one or more processors are configured to:
estimate a travel direction of an endoscope in the endoscopic examination based on the site information and the timing information of each of the plurality of endoscopic images; and
determine the position of the plurality of the first marks in the subregion based on the estimated travel direction.

8. The medical assistance system according to claim 2, wherein
the one or more processors are configured to:
display the organ model image provided with the first mark and the endoscopic image provided with the second mark at the same time.

9. The medical assistance system according to claim 2, wherein
the one or more processors are configured to:
display the endoscopic image provided with the second mark; and
upon receiving a user operation of switching a display image, display the organ model image provided with the first mark in place of the endoscopic image.

10. The medical assistance system according to claim 1, wherein
the presence information includes information indicating that there is a spot to be biopsied or information indicating that there is already a biopsied spot.

11. The medical assistance system according to claim 1, wherein
the one or more processors are configured to:
determine positions of the plurality of the first marks such that the plurality of the first marks do not overlap in the subregion.

12. A medical assistance method, wherein an organ model image includes a plurality of subregions associated with a plurality of sites of an organ, comprising:
acquiring presence information indicating that there is a biopsy spot in an endoscopic image;
acquiring site information indicating a site included in the endoscopic image;
acquiring timing information indicating an imaging time of the endoscopic image;
providing a first mark indicating that a biopsy has been performed or a biopsy should be performed to the subregion in the organ model image associated with the site indicated by the site information based on the presence information and the site information;
when there are a plurality of the endoscopic images of a different biopsy spot in the same site, determining positions of a plurality of the first marks in the subregion associated with the same site based on an imaging order; and
displaying the organ model image provided with the plurality of the first marks.

13. A recording medium having embodied thereon a program, wherein an organ model image includes a plurality of subregions associated with a plurality of sites of an organ, comprising computer-implemented modules including:
a module that acquires presence information indicating that there is a biopsy spot in an endoscopic image;
a module that acquires site information indicating a site included in the endoscopic image;
a module that acquires timing information indicating an imaging time of the endoscopic image;
a module that provides a first mark indicating that a biopsy has been performed or a biopsy should be performed to the subregion in the organ model image associated with the site indicated by the site information based on the presence information and the site information;
a module that determines, when there are a plurality of the endoscopic images of a different biopsy spot in the same site, positions of a plurality of the first marks in the subregion associated with the same site based on an imaging order; and
a module that displays the organ model image provided with the plurality of the first marks.
